(19) United States
(12) Patent Application Publication
(10) Pub. No.: US 2021/0253747 A1
(43) Pub. Date: Aug. 19, 2021

(54) COMPOSITIONS OF SOLUBLE BETA GLUCAN AND RELATED METHODS

(71) Applicant: KEMIN INDUSTRIES, INC., Des Moines, IA (US)

(72) Inventors: Geoffrey Horst, Grosse Pointe Farms, MI (US); Ye Lao, West Des Moines, IA (US)

(21) Appl. No.: 17/180,049

(22) Filed: Feb. 19, 2021

Related U.S. Application Data
(60) Provisional application No. 62/978,411, filed on Feb. 19, 2020.

(51) Int. Cl. C08B 37/00; A61K 9/08; A61K 9/16; A61K 31/716

(52) U.S. Cl. CPC C08B 37/0024; A61K 31/716; A61K 9/1682; A61K 9/08

(57) ABSTRACT

The present invention relates to a dried, resoluble beta-glucan and methods related to the same. Another aspect of the invention relates to the method of making the resoluble beta-glucan comprising the steps of combining an insoluble beta-glucan with water to form a suspension, followed by the addition of a strong base to form a solution. The solution is then dried to a moisture content of at least 20% by weight or less.

### RELATED APPLICATION

This application claims the benefit of priority to U.S. Provisional Patent Application No. 62/978,411, filed Feb. 19, 2020, entitled “COMPOSITIONS OF SOLUBLE BETA GLUCAN AND RELATED METHODS,” the entire disclosure of which is hereby incorporated by reference in its entirety.

### BACKGROUND OF THE INVENTION

Beta-glucans are polysaccharides (carbohydrates) produced by a wide variety of organisms including bacteria, fungi, yeasts, algae, protists, lichens, and plants such as oats and barley. Many varieties are taken as herbal medicines, to prevent and treat cancer, lower cholesterol, human immunodeficiency virus (HIV), and diabetes, and to increase the immune system function.

Chemically, beta-glucans are simply polysaccharides consisting of only connected glucose units. Different organisms produce different forms of beta glucan with different linkage connections between the glucose units. The type of linkages between the glucose units as well as the molecular weight of the molecules result in vastly different physical characteristics like solubility in water and biological interactions with immune cells.

For example, yeast produce beta β-(1-3)/β-(1-6) glucans in their cell walls. This means the glucose units are connected between the 1^{st} and 3^{rd} carbons “β-(1-3)” and occasionally between the 1^{st} and 6^{th} carbons “β-(1-6)”. Other sources of β-(1-3)/β-(1-6) glucan include seaweed, and various mushrooms such as lingzhi, shiitake, chaga, and maitake and resemble a branching structure with straight β-(1-3) sections and β-(1-6) branches coming off the sides. In oats and barley, glucose monomers are linked via β-(1-4) and β-(1-3) glycosidic bonds.

Paramylon is a linear (unbranched) β-(1,3)-glucan polysaccharide polymer with high-molecular weight. Euglena can accumulate large amounts of this reserve polysaccharide as granules in the cytoplasm up to 95% of the cell mass, when grown in the presence of adequate carbon sources under heterotrophic growth conditions.

Paramylon has a very high level of crystallinity in the native state (about 90%). The crystallinity of paramylon is due to higher order aggregates of microfibrils. Its high crystallinity is an advantage, in that, paramylon granules can be isolated at very low cost and in an efficient manner by simply disrupting the cells and purifying the granules by successive washing with low-concentration detergent. Paramylon has no solubility in water at ambient temperatures, and its molecular weight is estimated to be larger than 500 kDa. This lack of solubility in aqueous media is a major obstacle to its clinical utilization.

There is therefore a need in the industry for an effective means of solubilizing paramylon and other insoluble sources of beta glucan.

### BRIEF SUMMARY OF THE INVENTION

The present invention relates to a dried, resoluble beta-glucan and methods related to the same. Another aspect of the invention relates to the method of making the resoluble beta-glucan comprising the steps of combining an insoluble beta-glucan with water to form a suspension, followed by the addition of a strong base to form a solution. The solution is then dried to a moisture content of at least 20% by weight or less.

According to at least one embodiment of the present invention, the paramylon or other insoluble beta-glucan (or source thereof) is first treated with a strong base of at least 0.5N concentration, followed by drying to a moisture content of less than about 10% by weight. The final product forms a soluble/colloidal solution when added to water.

### DETAILED DESCRIPTION OF THE FIGURE

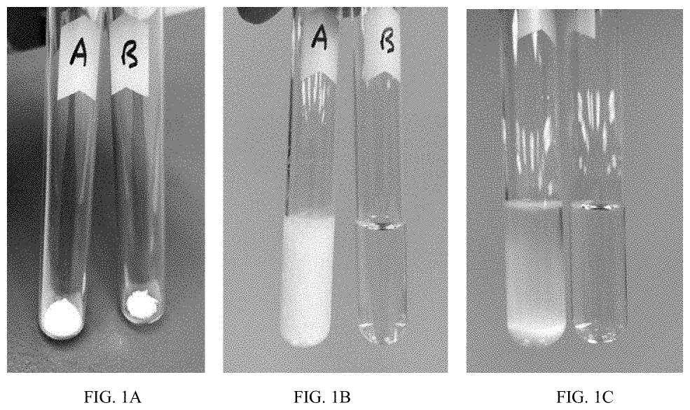

FIGS. 1A, 1B, and 1C are photographs showing the results of a solubility experiment comparing native paramylon granules (sample A) to the dry powder composition product (sample B) prepared in Example 1.

### DETAILED DESCRIPTION OF THE INVENTION

The invention relates to the dried, resoluble paramylon and the processes and methods related to the same. The paramylon starting material used in the invention includes not just pure paramylon but sources thereof, including Euglena gracilis and all or some of the cell components thereof. These cell components include, but are not limited to, lipids, proteins, carbohydrates (other than beta glucan), minerals, and vitamins. The invention is also intended to include other potential natural and synthetic sources of paramylon. The invention is also useful in solubilizing other insoluble types of beta-glucan, including but not limited to beta-glucans having β-(1,3) linkages and a high degree of polymerization.

The paramylon or other insoluble beta-glucan is first mixed with water to form a suspension. In one embodiment, the beta-glucan is preferably added to the water to form a 1-50% by weight suspension. In another embodiment, the beta-glucan is added to the water in an amount to form a 2-10% by weight suspension. The suspension is preferably stirred to help form a homogenous suspension.

A strong base is next slowly added to the beta-glucan suspension in an amount and a concentration sufficient to unravel the beta-glucan and form a solution having a concentration of at least 0.01N strong base. According to at least one embodiment, the strong base has a concentration of 0.1N or higher. In another embodiment, the strong base has a concentration of between about 1-15 N. In another embodiment, the strong base is added to the suspension to form a solution of between about 25-75 g/L paramylon (insoluble beta-glucan).

The aqueous suspension/strong base are mixed/stirred/agitated for a period of time sufficient to ensure complete dissolution of the beta-glucan. This amount of time can vary according to the concentration of beta-glucan, the concentration of base, the type of strong base, etc. Persons skilled in the art would readily understand the types of strong bases that would be useful in the invention including, but not limited to, alkaline hydroxides.

The aqueous suspension/strong base composition may include other ingredients, so long as they are compatible with the other ingredients. Such ingredients would be well-known to persons skilled in the art including, but not limited to, pH buffers, minerals, drying and processing aids such as gums, proteins, emulsifiers, lubricants, fillers, carbohydrates, silica, coloring agents, flavorings, vitamins, other active ingredients, etc.

Once dissolution is complete, the solution is dried to form a dried, resoluble paramylon/insoluble beta-glucan resoluble composition. Any form of drying known in the art may be used for this step including, but not limited to, air drying, drum drying, freeze drying, spray drying, microwave drying, convective drying, conductive drying, and combinations thereof. In one embodiment, the solution is dried to 20% by weight or less moisture. In another embodiment, the solution is dried to a moisture content of about 10% by weight or less. The resoluble composition of the present invention should be capable of passing through a 100 micron mesh and not precipitate for a time period of at least one hour.

The final composition may be redissolved to form a soluble/colloidal water-based solution having a final concentration of at least about 1 g/L beta-glucan. In one embodiment, the final concentration of beta-glucan in the redissolved solution is between about 5-15 g/L beta-glucan. Before redissolving the dried composition, the composition is ground using conventional mechanical grinding/milling/pulverizing methods, including but not limited to, grinding with a mortar and pestle.

The compositions of the present invention may optionally be combined with a pharmaceutically acceptable carrier that may include one or more carriers or excipients, such as fillers, diluents, binders, lubricants, and disintegrants. Such ingredients and their relative amounts to be included are well known to persons skilled in the art.

The following examples are offered to illustrate but not limit the invention. Thus, it is presented with the understanding that various formulation modifications as well as method of delivery modifications may be made and still are within the spirit of the invention.

### Example

Five grams of substantially pure paramylon granules (>95% beta glucan), extracted from Euglena gracilis, is added to 90 mL of water and stirred for 5 minutes to create a homogenous fine white particle suspension. Ten mL of a concentrated NaOH solution (10N) is added dropwise over 30 minutes while under constant stirring to create a solution with a final concentration of NaOH of 1N and 50 g/L concentration of paramylon. With increasing amounts of NaOH, the paramylon granules begin to unravel their crystalline structure and the solution becomes progressively more transparent with no visible paramylon granules and the solution becomes more viscous. After the final drops of NaOH are added, the solution is mixed for at least 60 more minutes to insure complete dissolution. This fully dissolved solution is now freeze dried (lyophilization) using nominal operating parameters (e.g. initial freeze temperature of less than −30 deg C., and less than 500 mTorr). The material is dried to less than 10% moisture and this product is an example of the claimed composition product.

The final criteria for the claimed composition is the ability for the dry product to redissolve into a soluble/colloidal water-based solution at an example concentration of 10 g/L. Before redissolving in water, the dry product is ground into a powder using any mechanical grinding/milling/pulverizing methods, in this case a mortar and pestle. As an example of demonstrating resolubility, a 0.05 gram sample of the dry powdered product is added to 5 mL of room temperature water in a test tube (i.e. 10 g/L paramylon concentration) and shaken for 60 seconds.

The definition of a soluble/colloidal solution in this invention is measured by either of two methods to demonstrate that the re-dissolved/colloidal paramylon does not settle out of solution.
1) The 5 ml sample after mixing is measured for absorbance at 600 nM (after setting a zero reading with pure water) in a spectrophotometer with a reading measurement height of at least 1 cm above the bottom of the tube. If the absorbance reading does not decrease by more than 25% after 60 minutes, then this indicates that the beta glucan has not settled out of solution.
2) If the total dissolved solids concentration of a sample collected from the top 2 mL of a 5 mL sample tube after 60 minutes is not less than 75% of the total dissolved solids concentration of the bottom 2 mL of the sample tube, then this is another indication that the beta glucan has not settled out of the solution.
   Solubility experiment comparing native paramylon granules (sample A) to the dry powder composition product (sample B) prepared in Example 1. FIGS. 1A, 1B, and 1C are photographs showing illustrating the results with sample A shown in the left test tube and sample B shown in the right test tube in each instance.

### Absorbance-Based Quantitative Data:

Sample A: Initial absorbance (@600 nM)=1.950; absorbance after 60 minutes=1.045
Absorbance at 60 min/Initial absorbance=1.045/1.950=0.535
Decreased by more than 25% after 60 minutes,
therefore defined as NOT SOLUBLE
   Sample B: Initial absorbance (@600 nM)=0.033; absorbance after 60 minutes=0.030
0.030/0.033=0.909
Decreased by less than 25% after 60 minutes, therefore defined as SOLUBLE

### Total Dissolved Solids Quantitative Data:

Solids determined by aspirating 2 mL from the top layer of the 5 mL tube sample (Top) or bottom 2 mL layer (Bottom) and drying in an oven at 105 deg C. with the net dish weight difference representing the total dissolved solids in each 2 mL aliquot
Sample A: Solids from top 2 mL layer=0.7 mg; solids from bottom 2 mL layer=55.3 mg
Top/bottom solids=0.7/55.3=0.012
Top sample is less than 75% of the bottom sample weight (only 1.2%),
therefore defined as NOT SOLUBLE
   Sample B: Solids from top 2 mL layer=39.1 mg;
solids from bottom 2 mL layer=39.4 mg
Top/bottom solids=39.1/39.4=0.992
Top sample is more than 75% of the bottom sample weight (99.2%),
therefore defined as SOLUBLE

It should be appreciated that minor dosage and formulation modifications of the composition and the ranges expressed herein may be made and still come within the scope and spirit of the present invention.

Having described the invention with reference to particular compositions, theories of effectiveness, and the like, it will be apparent to those of skill in the art that it is not intended that the invention be limited by such illustrative embodiments or mechanisms, and that modifications can be made without departing from the scope or spirit of the invention, as defined by the appended claims. It is intended that all such obvious modifications and variations be included within the scope of the present invention as defined in the appended claims. The claims are meant to cover the claimed components and steps in any sequence which is effective to meet the objectives there intended, unless the context specifically indicates to the contrary.

The foregoing description has been presented for the purposes of illustration and description. It is not intended to be an exhaustive list or limit the invention to the precise forms disclosed. It is contemplated that other alternative processes and methods obvious to those skilled in the art are considered included in the invention. The description is merely examples of embodiments. It is understood that any other modifications, substitutions, and/or additions may be made, which are within the intended spirit and scope of the disclosure. From the foregoing, it can be seen that the exemplary aspects of the disclosure accomplish at least all of the intended objectives.

1. A composition comprising dried, resoluble beta-glucan that when dissolved in water is capable of passing through a 100 micron mesh and does not precipitate for a time period of at least one hour, wherein the composition comprises a source of insoluble beta-glucan.

2. The composition of claim 1 whereby the source of insoluble beta-glucan comprises cellular components of Euglena gracilis.

3. The composition of claim 1 wherein the source of insoluble beta-glucan is paramylon.

4. The composition of claim 1 which contains at least 1 g/L beta-glucan.

5. A method of forming a dried, resoluble beta-glucan comprising the steps of:
combining a source of insoluble beta-glucan with water to form a suspension;
adding a strong base to the suspension to form a solution; and
drying the solution to form the dried, resoluble beta-glucan.

6. The method of claim 5 herein the insoluble beta-glucan and the water are stirred to form a homogenous suspension.

7. The method of claim 5 wherein the strong base has a concentration of at least 0.01N.

8. The method of claim 5 wherein the strong base has a concentration of at least 1N.

9. The method of claim 5 wherein the concentration of the insoluble beta-glucan in the solution is 25-75 g/L.

10. The method of claim 5 wherein the suspension and strong base are mixed, stirred, or agitated for a period of time sufficient to provide complete dissolution of the beta-glucan.

11. The method of claim 5 wherein one or more other ingredients are added to the solution, whereby the one or more other ingredients are selected from the group consisting of pH buffers, minerals, drying and processing aids such as gums, proteins, emulsifiers, lubricants, fillers, carbohydrates, silica, coloring agents, flavorings, vitamins, and other active ingredients.

12. The method of claim 5 wherein the drying step is selected from one or more methods selected from the group consisting of air drying, drum drying, freeze drying, spray drying, microwave drying, convective drying, and conductive drying.

13. The method of claim 5 wherein the solution is dried to 20% by weight or less moisture.

14. The method of claim 5 wherein the solution is dried to 10% by weight or less.

15. The method of claim 5 wherein the dried, resoluble beta-glucan comprises at least about 1 g/L beta-glucan.

16. The method of claim 5 wherein the dried, resoluble beta-glucan comprises between about 5-15 g/L beta-glucan.

17. The method of claim 5 further including the step of grinding the dried, resoluble beta-glucan.

18. The method of claim 5 further including the step of combining the dried, resoluble beta-glucan with a pharmaceutical carrier.
